(19) United States
(12) Patent Application Publication
(10) Pub. No.: US 2010/0100055 A1
(43) Pub. Date: Apr. 22, 2010

(54) Devices for Superficial Femoral Artery Intervention

(75) Inventor: Jihad Mustapha, Ada, MI (US)

Correspondence Address: Jonathan P. O''Brien, Ph.D.;Honigman Miller Schwartz and Cohn, 350 East Michigan Avenue, Suite 300, KALAMAZOO, MI, 49007, US

(73) Assignee: TD.JAM Medical Technologies , LLC, Ada, MI (US)

(21) Appl. No.: 12/470,026

(22) Filed: May 21, 2009

Related U.S. Application Data
(60) Provisional application No. 61/055,177, filed on May 22, 2008.
(60) Provisional application No. 61/077,647, filed on Jul. 2, 2008.

(51) Int. Cl. A61M 25/00; A61M 25/14

(52) U.S. Cl. 604/246; 604/525

(57) ABSTRACT

This invention provides various sheaths, guards, and introducer assemblies for accessing a remote area or region of an artery, such as for delivery and/or deployment of an interventional device, stent or the like. The sheath, guard, and introducer assembly of the present inventions provide varying degrees of flexibility and support, enhanced guidance and control, and blood perfusion apertures to ameliorate the intervention of the superficial femoral artery.

### FIELD OF THE INVENTION

The present invention relates to devices for treatment of arteries or vessels and the like and, more particularly, for treatment of a superficial femoral artery of a patient.

### BACKGROUND OF THE INVENTION

Endovascular treatment of peripheral vascular occlusive disease has emerged as a preferred and viable alternative for patients over conventional surgery. However, endovascular recanalization of the superficial femoral artery (SFA) faces a number of challenges. This is because the SFA is the longest artery in the human body and has two major flexion points, at the hip at its proximal end and at the knee joint at its distal end. Currently, there is a great need to develop safer and more effective procedures for intervention of the SFA and other blood vessels.

### SUMMARY OF THE INVENTION

The present invention provides various sheaths, guards, and introducer assemblies for accessing a remote area or region of an artery, such as for delivery and/or deployment of an interventional device, stent or the like. The sheath, guard, and introducer assembly of the present inventions provide varying degrees of flexibility and support, enhanced guidance and control, and blood perfusion apertures to ameliorate the intervention of the superficial femoral artery.

According to an aspect of the present invention, an interventional sheath is provided for delivering an interventional device to a distal location in a vessel that is remote from a patient entry location and includes portions having varying degrees of flexibility. For example, the sheath may include a proximal portion having a first degree of flexibility, an intermediate portion having a second degree of flexibility, and a distal portion having a third degree of flexibility. The proximal portion is at a proximal region of the sheath near the patient entry location, while the intermediate portion is at an intermediation region of the sheath, and the distal portion is at a distal region of the sheath which is remote from the patient entry location. The second degree of flexibility is greater than the first degree of flexibility, while the third degree of flexibility is greater than the second degree of flexibility.

Optionally, the sheath may include circular or coiled wires at the proximal portion, the intermediate portion and the distal portion, with the circular wires having a greater density or reduced spacing at the proximal portion than at the intermediate portion. The circular wires also have a greater density at the intermediate portion than the distal portion.

According to another aspect of the present invention, the sheath may further comprise at least one side aperture at the side wall of the sheath. The side apertures may be openable and closable via movement of a wire enclosed within and running along the sheath wall, with the wire having at least one aperture therealong that is alignable with the side aperture at the side wall to open the side aperture. The wire is movable relative to the sheath which can be used to move the wire aperture away from the sheath wall aperture to close or obstruct the sheath side wall aperture. When the apertures of the wire and sheath are aligned, it will allow blood to flow through to the distal portion of the sheath and this inflow and outflow will allow better visualization of the interventional procedure.

According to another aspect of the present invention, the sheath may comprise at least one side aperture at the side wall of the sheath which may be openable and closable via movement of an inner circular member enclosed within the sheath lumen and running along the sheath wall. The inner circular member may comprise at least one aperture or open segment therealong that is alignable with the sheath aperture in the side wall to allow blood to circulate through the sheath and the inner circular member. The inner circular member is movable relative to the sheath which can be moved to a position that closes or obstructs the sheath wall apertures. When the apertures of the inner circular member and sheath are aligned, it will allow blood to flow through to the distal regions of the vessels which will protect those distal regions and allow better visualization during the interventional procedure.

According to another aspect of the present invention, a guard device may be attached to the sheath. The guard device comprises different one way valves that can be used to assist with the insertion of a sheath or catheter to access a remote or distal area of a vessel or artery. The guard device reduces the backward force generated along the sheath or catheter during the delivery of an interventional device to the remote portion of the vessel. The guard device limits the backward force reaching the operator's hands and, thus, provides enhanced control during delivery of the intervention device. The two one-way valves of the guard device allow the guard to have multiple roles or purposes. One of valves may be used to deploy a sheath during straight forward interventions that do not require extra backup support. The second or hollow lumen of the other one way valve allows for interventions needing extra backup support. The additional control from the hollow lumen one way valve is created from the constant and simultaneous hold on a catheter and sheath during insertion to the remote location of a vessel.

According to another aspect of the present invention, an introducer device may be attached to the distal tip of an interventional sheath to aid in the insertion of the sheath to remote regions of a vessel. The introducer device comprises a plurality of tapered angle portions, perfusion apertures, a tip, a base, and a fastening member. The introducer device with its variable flexibility and hardness can be adapted to attach to any sheath size and can assist and reduce resistance during interventions crossing Aortoilliac junctions. The distal tip of the introducer device has a wide curved tip that is medially angled to accommodate the variable angles of Aortoilliac junctions and to deflect blood flow from the vessels. The perfusion apertures of the introducer device allow symmetrical flow of the blood or liquid through the introducer device and away of the vessel wall.

These and other objects, advantages, purposes and features of the present invention will be more fully understood and appreciated by reference to the below description of the preferred embodiments and the drawings.

### BRIEF DESCRIPTION OF THE DRAWINGS

FIG. 1 is a plain view of an interventional sheath having varying degrees of flexibility and support in accordance with the present invention;

FIG. 2 is a cross sectional view of a portion of the interventional sheath of FIG. 1;

FIG. 3 is a side view of another portion of the sheath of FIG. 1;

FIG. 4 is a cross sectional view of another embodiment of the present invention showing a movable wire element enclosed within a sheath wall which opens and closes apertures on the sheath;

FIG. 5 is a sectional and partial perspective view of the sheath wall, showing the apertures formed therethrough, and showing the apertures as open (white) when the apertures of a wire element are aligned with the apertures of the sheath wall;

FIGS. 6 and 6a are side views of the wire element disposed within a sheath wall channel which is used for opening and closing the sheath apertures of the present invention, as shown the wire element is in the open position;

FIG. 7 is a sectional and partial perspective view of the sheath wall, showing the apertures formed therethrough, and showing the apertures as closed (dark) when the apertures of a wire element are not in alignment with the apertures of the sheath wall;

FIGS. 8 and 8a are side views of the wire element disposed within the sheath wall channel which is used to open and close the sheath apertures of the present invention, as shown the wire element is in the closed position relative to the sheath;

FIG. 9 is a perspective view showing a portion of the wire element and the relative positions of the wire element apertures used for opening and closing the apertures of the sheath;

FIG. 10 is a cross sectional view of another invention and embodiment that shows a sheath and an inner circular segment, showing the apertures through the sheath wall which are alignable with the inner circular member by adjusting the handle located at the proximate portion of sheath;

FIG. 11 is a plan view of another invention and embodiment that shows a sheath and inner circular segment, showing the apertures through the sheath wall which are alignable with the perfusion aperture of the inner circular member by rotating the control dial;

FIG. 12 is a cross-sectional view of another invention and embodiment that shows a sheath and inner circular segment, showing the apertures through the sheath wall which are resiliently and securely alignable with the perfusion aperture of the inner circular member by rotating the control dial to the desirable position;

FIG. 13 is a plan view of the control dial fixed to the inner circular member and sheath which may be used to resiliently align the apertures of the sheath wall and the inner circular member;

FIG. 14 is a plan view of another invention and embodiment that shows a guard and valve assembly used to enhance operational control during procedures delivering interventional sheaths, catheters or devices, as shown a sheath and catheter are inserted through the valves on the guard device and then disposed along and into a remote area of a vessel;

FIG. 15 is a top view of the guard and valve assembly of the interventional delivery system of FIG. 14;

FIG. 16 is a cross sectional view of the guard and valve assembly showing a sheath inserted through and frictionally engaging a one way valve on the guard;

FIG. 17 is a top view of the guard and valve assembly showing the valves adjusted and located to provide a different one way valve for insertion of a catheter;

FIG. 18 is a cross sectional view of the guard and valve assembly of FIG. 17 with a interventional catheter disposed through the hollow lumen one way value and the threaded region of the guard frictionally engaging the soft sheath wall;

FIG. 19 is another plan view of the guard and the interventional device delivery system of FIG. 14, showing the reactionary forces along the sheath and delivery system during operation of the system and in accordance with the present invention;

FIG. 20 is a side view of an introducer device used to enhance operational control during procedures delivering interventional sheaths, catheters or devices;

FIG. 21 is a side view of the introducer device of FIG. 20 that shows a flexible guide wire that is capable of being retained within the introducer device and sheath to assist into the intervention of a remote area of a vessel;

FIG. 22 is another side view of the introducer device of FIG. 20 that shows a guide wire retained within the lumen of a flexible introducer device and sheath where the guide wire where is used to adjust the shape of the introducer device; and

FIG. 23 is a plan view of the introducer device of FIG. 20 that shows the introducer device fastened to the distal end of a sheath and guard which are used to enhance operational control during procedures delivering interventional devices.

### DESCRIPTION OF THE PREFERRED EMBODIMENTS

### Interventional Sheath with Adjustable Support

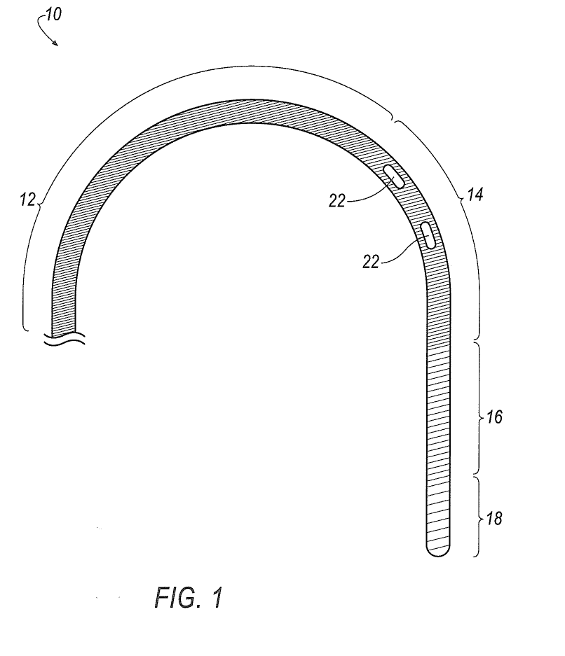

An interventional sheath or sheath assembly 10 is provided to deliver a device inside a vessel of a patient for endovascular intervention (FIG. 1). The interventional sheath 10 is well suited for use in delivering and/or deploying a device (such as a chronic total occlusion (CTO) device, an intervention balloon, a stent and/or the like) in the superficial femoral artery and to deliver a device at a location that is below the knee and along the superficial femoral artery of the patient. The interventional sheath 10 provides varying degrees of flexibility and support along the sheath 10 to assist the physician in guiding the sheath 10 through the vessel or artery, while providing the appropriate amount of support. In the illustrated embodiment of FIG. 1, the interventional sheath 10 provides three sections or portions having different degrees of flexibility and support: a proximal portion 12, an intermediate portion 14, and a distal portion 16. A fourth portion or section provides an atraumatic tip 18 at the end of the sheath or system to assist in guiding the sheath along and inside the artery or vessel, as discussed below.

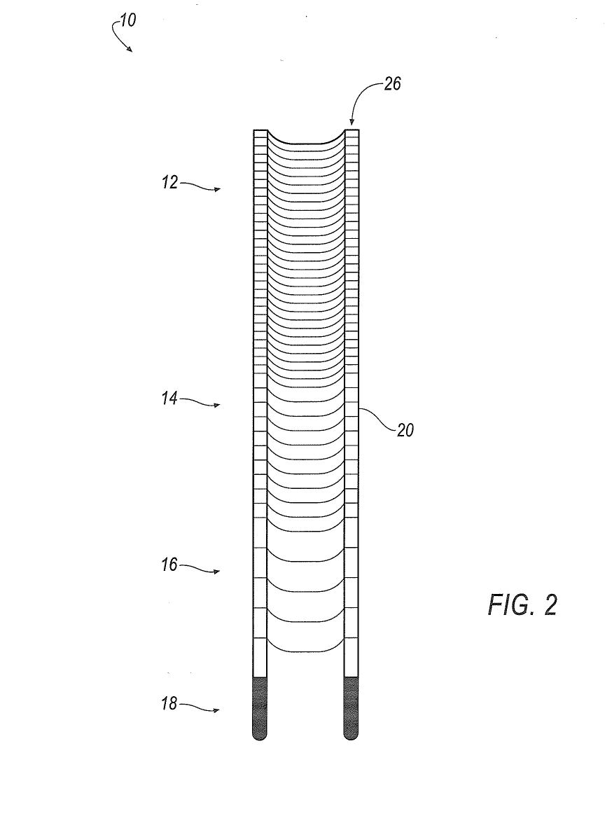

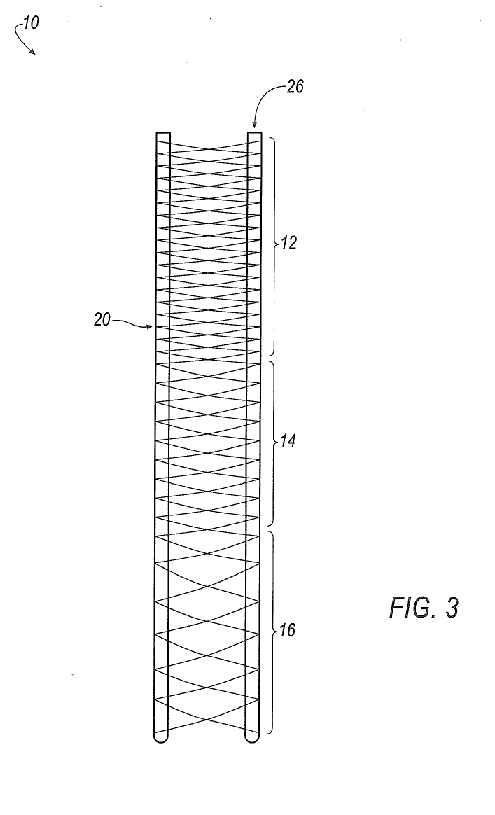

As can be seen in FIGS. 2 and 3, the sheath 10 provides greater support and reduced flexibility at the proximal portion 12, while providing a varying degree of support and flexibility at the intermediate portion 14 and a greater degree of flexibility and reduced support at the distal portion 16. The sheath 10 includes one or more circular or coiled wires 20 contained within the sheath wall 26, with the circular wires 20 having different densities or different number of wires per centimeter to provide the varying degrees of flexibility and support. The density or spacing between the circular wires 20 becomes reduced or closer at the proximal portion 12 relative to the intermediate portion 14 and distal portion 16. Along the sheath 10, the spacing between circular wires 20 transitions between the proximal portion 12, intermediate portion 14, and distal portion 16. This transition provides greater support towards the proximal portion 12 and the greater flexibility towards the intermediate portion 14 and distal portions 16. As can been seen in FIG. 2, the circular wire 20 is terminated before the atraumatic tip 18 of the sheath 10 to allow the tip of the sheath to be flexible and safe. In one example, the atraumatic tip 18 is composed of soft, glide material to assist in introduction of the sheath to distal portions of the vessel.

Thus, the proximal portion 12 of the sheath 10 is constructed to provide extra support at the proximal region of the vessel, which is suitable to deliver an interventional device across the horn of the aortoilliac junction. The condensed circular wires 20 at the proximal portion 12 allow for slight flexibility in the sheath, while providing enhanced support needed for that particular region.

As illustrated in the FIGS. 2 and 3, the intermediate portion 14 provides a transitional portion or region between the proximal portion 12 and the distal portion 16. The intermediate portion 14 provides moderate support along the sheath 10 relative to the proximate portion 12 and distal portion 16. The increased flexibility and decreased support of the intermediate portion 14 is accomplished by increasing the spacing along the circular wires 20, such as by about 50 percent (or more or less depending of the particular application of the interventional sheath) relative to the circular density or spacing of the proximal portion 12. As discussed above, there is a transition between the proximal portion 12 and the intermediate portion 14 that will steadily change the spacing or density between the circular wires 20 from the spacing along the proximal portion 12 to the spacing along the intermediate portion 14.

The distal portion 16 of the sheath 10 provides mild support at the distal region of the vessel, while providing enhanced flexibility due to a further reduction in the density of the circular wires 20, such as a reduction is about 50 percent of the intermediate portion 14. Thus, the distal portion 16 provides a soft or flexible sheath portion that still provides mild support while in the vessel, which is highly suited for intervention of the vessel without substantial risk of vessel trauma. Similar to the transition between the proximal portion 12 and the intermediate portion 14, the sheath provides a transition region between the intermediate portion 14 and the distal portion 16 by providing a gradual change in spacing between the circular wires 20.

The length of the sheath should be long enough for use in interventions in the superficial femoral artery of a patient and allow cross over interventions, and, thus, for example, the sheath 10 can vary from 10-160 centimeters long with preferred lengths at 40, 50, 60, 70, 80, 90, 100 centimeters. However, the sheath 10 may be constructed in different lengths depending on the particular application, and these different lengths of the sheath 10 should be included as part of the scope of the present invention. The three portions (although three portions of varying flexibility and support are shown in the drawings, a sheath may be constructed with two portions or more than three portions of varying flexibility and support while remaining within the spirit and scope of the present invention) of the sheath 10 having different degrees of flexibility and support provides a safer sheath and enhances support specifically around the horn of the aortoilliac junction, while providing the desired or needed amount of flexibility at the distal end of the sheath for guiding the sheath along and through the vessel.

The relative lengths of the various flexible/support portions of the sheath may be selected or varied to provide the desired support and flexibility to particular regions of the vessel to be intervened. For example, the proximal portion 12 of the sheath 10 may be selected to be longer to allow the extra support regions to cross into the contralateral illiac artery when inserted into a superficial femoral artery.

### Interventional Sheath with Perfusion Apertures

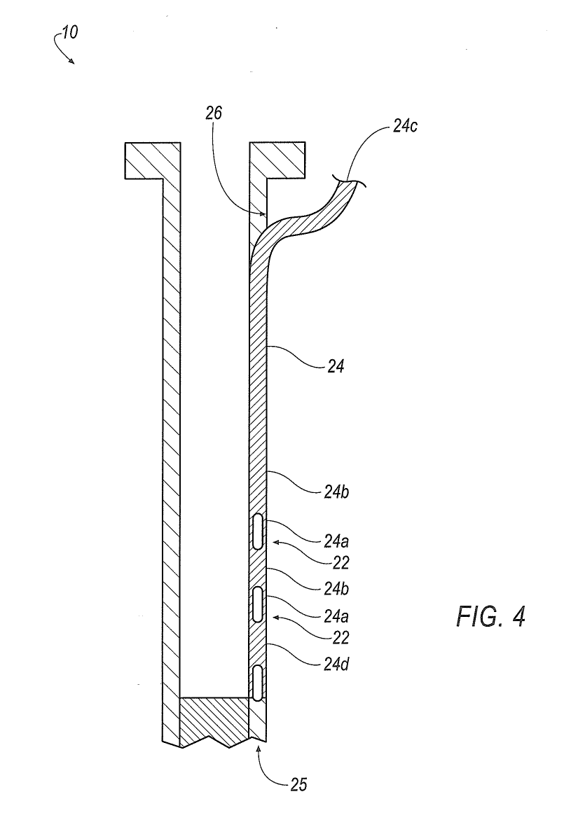

In one example, and as can be seen in FIGS. 4-12, an interventional sheath 10 may also include one or more apertures 22 through its side wall. The apertures 22 may be opened and closed as desired to allow for the flow of blood through the sheath into the distal locations of the vessel. The better inflow and outflow of fluid or blood along a vessel results in protection of distal regions of the vessel, enhanced control during intervention and better visualization by the physician performing the vessel intervention.

In the illustrated embodiment of FIG. 4, the apertures 22 are opened and closed via movement of a wire or wire element 24 that is disposed along and/or within a sheath wall 26 and is movable longitudinally therealong to open and close the apertures 22 of the sheath 10. In the illustrated embodiment of FIG. 4, the wire element 24 has one or more open segments or portions 24a and one or more closed or solid segments or portions 24b spaced therealong, such as two open segments 24a and two closed segments 24b, which correspond to the two apertures 22 spaced along the wall of the sheath 10. The spacing between the open segment 24a is approximately the same as the spacing between the closed segment 24b and the spacing between the apertures 22, such that movement of the wire along the sheath moves the segment 24a and 24b relative to the apertures 22 and functions to open or close the apertures 22 as desired.

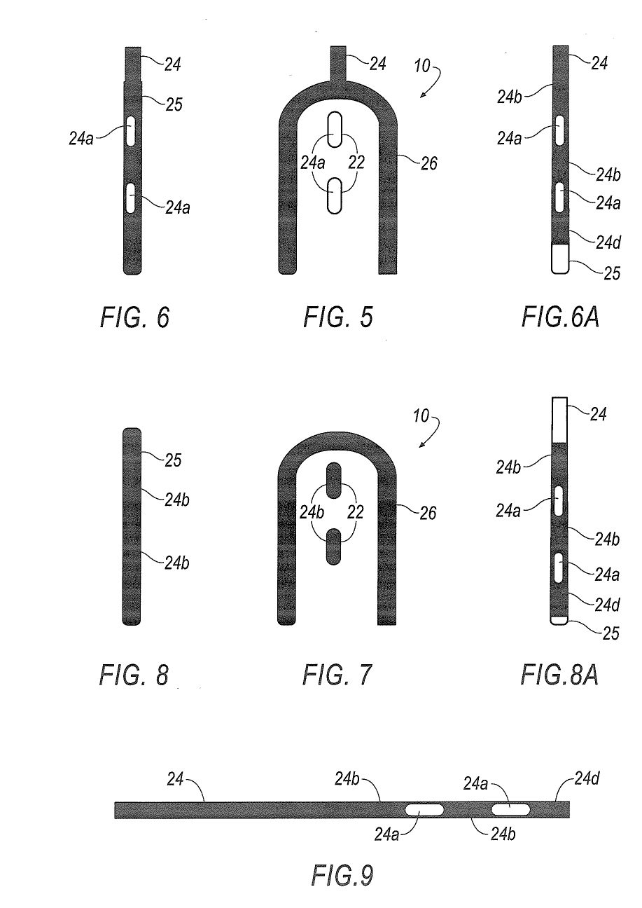

As can be seen in FIG. 4, the wire 24 is movably or adjustably disposed within a channel or housing 25 in the outer sheath wall 26. The wire 24 has a proximal end 24c that exits the sheath wall 26 at the proximate portion for access by a physician or operator, whereby the physician or operator may push and pull at the wire 24 to open and close the apertures 22 of sheath 10 as desired. As shown in FIGS. 5, 6, and 6a, when the apertures 22 are opened (such as when the open segments 24a of the wire element 24 are substantially aligned with apertures 22 of sheath 10) the sheath allows blood to flow through to distal sites. When such blood flow is less desired, the physician may readily move the wire 24 relative to the sheath wall 26 to close the apertures 22, and, thus, restrict blood flow through the sheath and/or vessel (FIGS. 7, 8, and 8a).

In the illustrated embodiment of FIG. 1, the apertures 22 are disposed in the side walls of the intermediate or moderate support portion 14 of sheath 10. However, the apertures may be disposed elsewhere along the sheath as desired and depending on the particular application intended for the sheath 10.

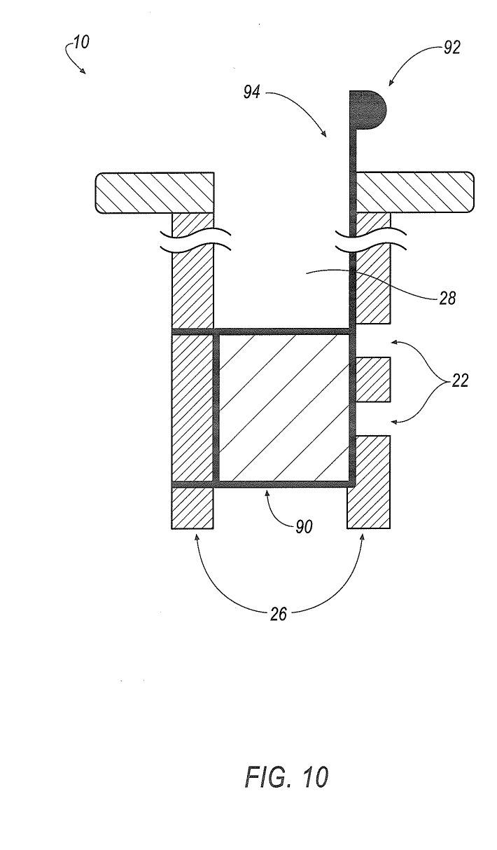

In the illustrated embodiment of FIG. 10, the sheath wall apertures 22 are opened and closed via movement of an inner circular member 90 that is inside the sheath lumen 28 and movably or adjustably disposed along and adjacent the sheath wall 26 and is movable longitudinally there along to open and close the apertures 22 of the sheath 10. The arrangement of the inner circular member 90 over the apertures 22 will effectively form a seal on the inside of the sheath wall 26. In the illustrated embodiment of FIG. 10, the inner circular member 90 is fixedly attached to a handle 94 and grip 92 that extend outwardly beyond the proximate portion of the sheath 10. The grip 92 and handle 94 allow the physician to effectively control and move the inner circular member 90 to a position over the sheath wall apertures 22 to obstruct, close or reduce blood flow to the distal portion of the vessel.

In the illustrated embodiment of FIG. 10, the apertures 22 are disposed and located at the proximate portion of sheath 10 in the sheath walls 26. However, the apertures 22 may be disposed elsewhere along the sheath as desired and depending on the particular application intended for the sheath 10.

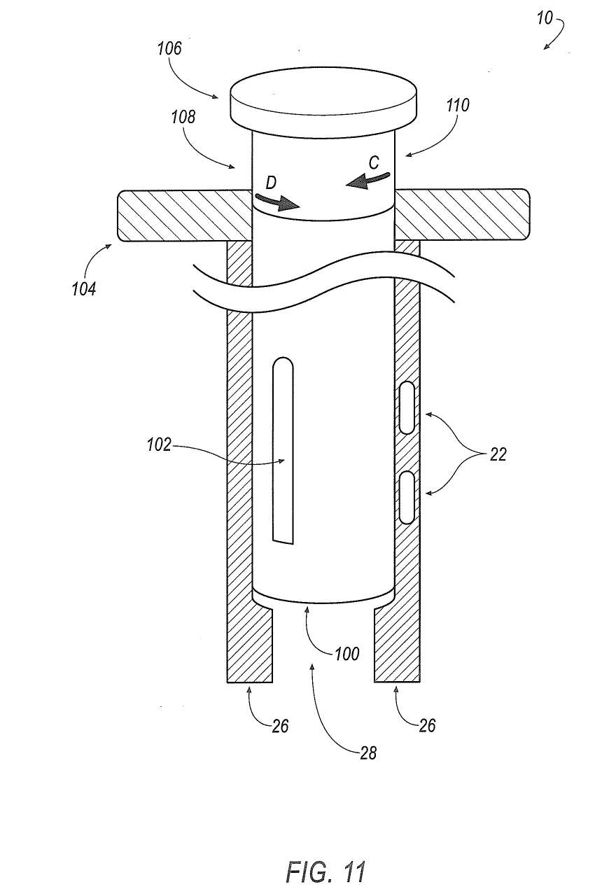

In the illustrated embodiment of FIG. 11, the sheath wall apertures 22 are opened and closed via rotation of a shutter or inner circular member 100 that is inside the sheath lumen 28 and disposed along and adjacent the sheath wall 26. In the illustrated embodiment of FIG. 11, the inner circular member 100 is fixed to a control dial 106 that extends outwardly beyond the proximate portion of the sheath 10. The control dial 106 allows the physician to effectively control and rotate the inner circular member 100 to a position where the sheath apertures 22 and the perfuse apertures 102 are separated and do not align which prevents or reduces the blood flow to the distal portion of the vessel.

As can be seen in FIG. 11, the inner circular member 100 is movably or adjustably disposed within a sheath lumen 28 and longitudinally abuts the sheath wall 26 which effectively forms a seal on the inside of the sheath wall 26. The inner circular member 100 has a proximal control dial 106 that is fixedly attached to the inner circular member 100 and exits the sheath wall 26 at the proximate portion for access by a physician or operator, whereby the physician or operator may rotate the inner circular member 100 to open and close the apertures 22 of sheath 10 as desired. As shown in FIG. 11, when the blood flow is less desired, the physician may readily rotate the inner circular member 100 towards the indicated closed mark 110 to obstruct or close the apertures 22, and, thus, restrict blood flow through the sheath and/or vessel. Alternatively, as shown in FIG. 11, when the blood flow is desired, the physician may rotate the inner circular member towards the indicated open mark 108. To ease the rotation of the inner circular member 100 relative to the sheath 10, the sheath walls 26 at the proximate portion or ends have an outwardly extending handles 104 that allow the physician to tightly hold the sheath 10 in place while the inner circular member 100 is rotated to the desired position.

In the illustrated embodiment of FIG. 11, the apertures 22 are disposed and located at the proximate portion of sheath 10 in the side of the sheath walls 26. However, the apertures 22 may be disposed elsewhere along the sheath as desired and depending on the particular application intended for the sheath 10.

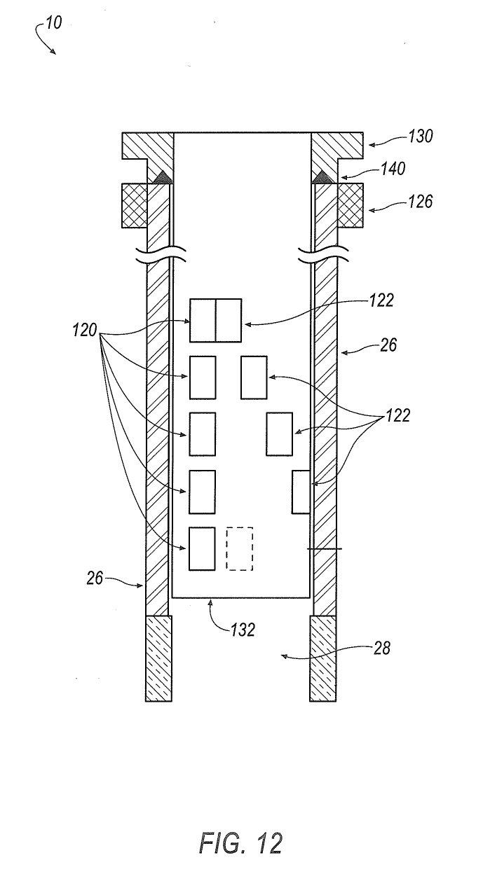

In the illustrated embodiment of FIG. 12, the sheath wall apertures 120 are opened and closed via rotation of a shutter or inner circular member 132 that is disposed within the sheath lumen 28 and adjacent the sheath wall 26 and is rotated to open and close the apertures 120 of the sheath 10. In the illustrated embodiment of FIG. 12, the inner circular member 132 is fixed to a control dial 130 that extends outwardly beyond the proximate portion of the sheath 10. The control dial 132 allows the physician to effectively control and move the inner circular member 132 to a position over the sheath wall apertures 120 to prevent or reduce blood flow to the distal portion of the vessel.

As can be seen in FIG. 12, the inner circular segment 132 is capable of controlled perfusion at different levels and locations for the sheath 10 and will assure that the sheath apertures 120 are open at a location where antegrade blood flow is present to allow distal perfusion of the treated target vessel. The inner circular member 132 is movably or adjustably disposed within a sheath lumen 28 and longitudinally abuts the sheath wall 26 which effectively forms a seal on the inside of the sheath wall 26. The sheath apertures 120 are placed in a straight vertical line or some other preferred arrangement on the sheath wall 26 while the perfusion apertures 122 of the abutting inner circular member 132 are located in a spiral pattern so only one sheath wall aperture 120 and only one perfusion aperture 122 line-up at time. The apertures 120 on the sheath wall 26 are opened one at a time by the rotation of the control dial 130. Alternatively, the sheath apertures 120 and the perfusion apertures 122 can be located in other complementary patterns to allow multiple sheath apertures 120 to be opened at a time including placing perfusion apertures 122 of the inner circular member 132 on opposing sides, as shown in dashed lines on FIG. 12, which allows for simultaneous distal perfusion of blood through more than one sheath wall aperture 120 at multiple and separate locations. As a result, the fixed rotation of the control dial 130 opens only those sheath wall apertures 120 intended by the operator to be opened at the same time and will close those other sheath wall apertures 120. Therefore, the current embodiments provide the operator with intimate hand control of the perfusion parameters and specifics of the sheath 10 during the intervention procedure.

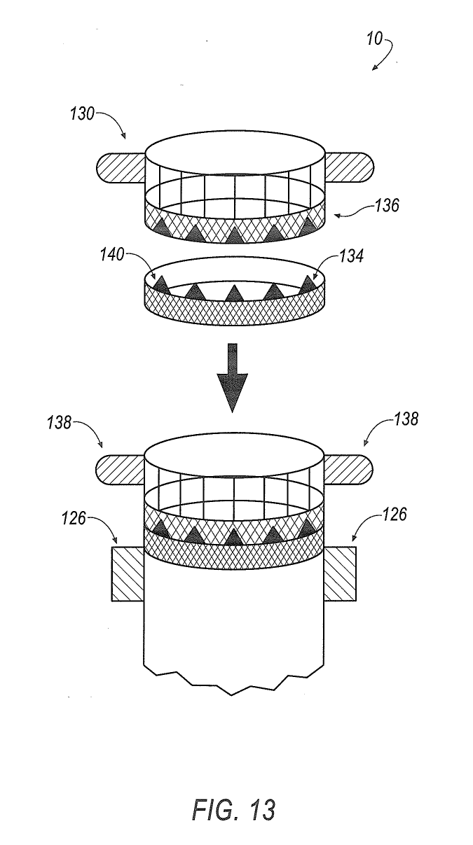

As illustrated in FIG. 13, the inner circular member 132 has a control dial 130 that is fixedly attached to both the proximal portion of the sheath 10 and the inner circular member 132. The control dial 130 exits the sheath wall 26 at the proximate portion for access by a physician or operator, whereby the physician or operator may rotate the inner circular member 132 to open and close the apertures 120 of sheath 10 as desired. As shown in FIGS. 12 and 13, the physician may readily move the inner circular member 132 relative to the sheath 10 to control the blood flow through the sheath and/or vessel. The control dial 130 has a top portion 136 and bottom portion 134 that engage and hold the inner circular member 132 in a fixed position relative to the sheath 10. The top portion 136 can be fixedly attached and connected to the circular member 132 through one of several different methods commonly used by those skilled in the art. As a result, rotation and movement of the top portion 136 simultaneously rotates the inner circular member 132. The bottom portion 134 of the control dial 130 is fixedly attached to the proximate portion of the sheath 10. The bottom portion 134 has detents 140 that engage and fit indents located on the top portion 136. The detents 140 are used to intentionally divide the rotation of the control dial 130 into discreet increments that align with the perfusion apertures 122 for controlled opening of the sheath wall apertures 120 and allow pre-determined perfusion of blood. The two portions of the control dial 130 at the proximal end of the sheath serves as a cock mechanism to keep the sheath apertures 120 and the perfusion apertures 122 aligned and secure and allow the operator to have sheath wall apertures 120 to be functional at one or several at a time. To ease the rotation of the inner circular member 132 relative to the sheath 10, the sheath walls 26 at the proximate portion or ends have an outwardly extending grip 126 that allows the physician to tightly hold the sheath 10 in place while the inner circular member 132 is rotated to the desired position by the protruding handles 138.

In the illustrated embodiment of FIGS. 12 and 13, the apertures 120 are disposed and located at the proximate portion of sheath 10 in the side walls 26. However, the apertures 120 may be disposed elsewhere along the sheath 10 as desired and depending on the particular application intended for the sheath. Optionally, and desirably, for the below the knee intervention, the first sheath aperture 120 is at 25 cm from the proximate end of the sheath 10. For sheaths with full ante grade capabilities, the first aperture 120 is 10 cm from the proximate end of the sheath 10.

### Interventional Guard Device

Optionally, an interventional sheath of the present invention may include a guard device disposed therealong for providing enhanced control of the sheath and reduced back forces to the operator or physician during vessel intervention.

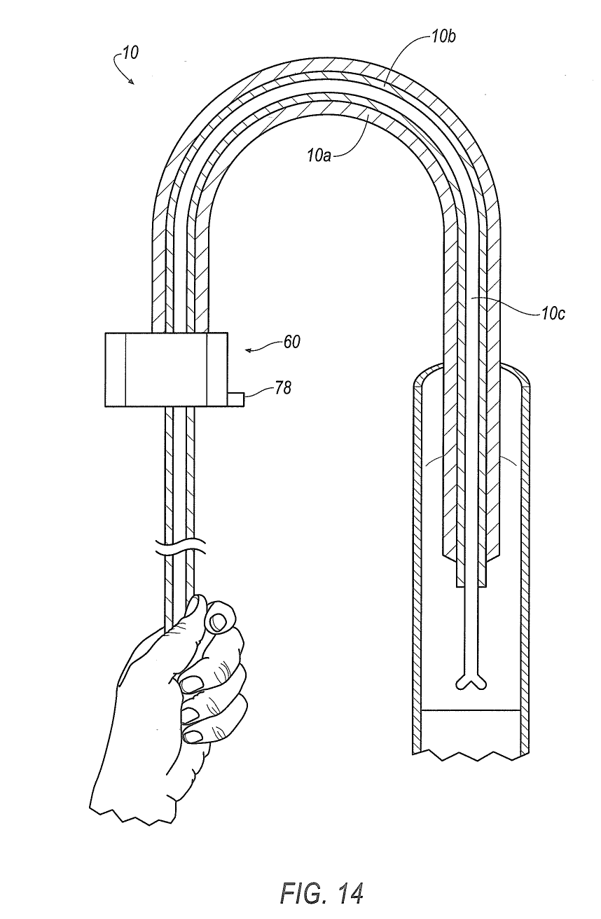

With references to FIG. 14, a guard device 60 may be provided along an interventional sheath, such as with the interventional sheath 10 described above. The guard device 60 may be disposed close and outside of the patient entry point at the proximate region of the sheath 10. The guard device 60 may be attached to the outer sheath portion 10a, along and through which an interventional catheter 10b is disposed, and within which an interventional device 10c is moved to access the distal region of the vessel (such as at a chronic total occlusion (CTO) site along the vessel or the like) for treatment of the vessel and/or for delivering and/or deploying an interventional device.

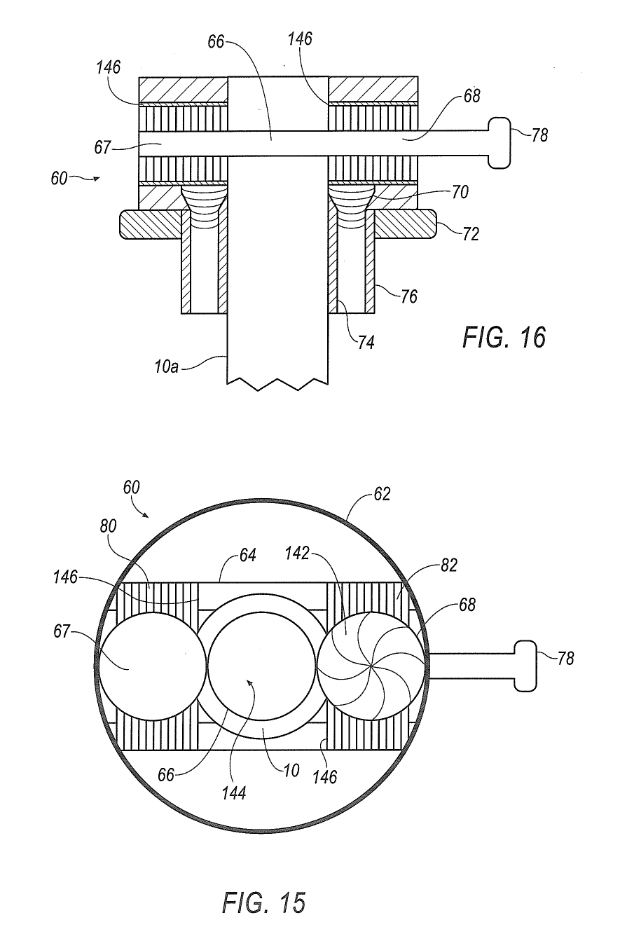

As shown in FIGS. 15 and 16, the guard device 60 includes an outer wall or housing 62 and a channel 64 extending across the top. The channel 64 houses compression springs 80 and 82, a one-way valve 66 and a hollow lumen one-way valve 68 that are movable along the channel 64. The guard device 60 is attached and capped to an outer sheath portion 10a, such as at or near the proximal end of the sheath 10. The one-way value 66 contains an open thru bore 144 and the hollow lumen one-way value 68 contains iris or diaphragm like control means 142 that frictionally engage and hold an interventional catheter 10b to the guard device 60 which is fixedly attached to the outer sheath portion 10a.

In the illustrated embodiment of FIGS. 16 and 18, the guard device 60 has a threaded region 70 and a collar 72 for rotating the threaded region 70. The threaded region 70 of the guard device 60 receives the outer sheath portion 10a therein and extends along the proximal portion of the sheath 10 and engages the soft internal wall 74 of the guard device 60. The guard device 60 also includes an outer support wall 76 that may be threaded such that rotation of threaded region 70 causes translational movement of threaded region 70 along the outer support wall 76. As can be seen with reference to FIGS. 16 and 18, rotation of the collar 72 causes the threaded portion 70 to move along the sheath 10 and to compress the soft inner wall 74 to secure the guard device 60 at the desired location along the sheath portion 10a.

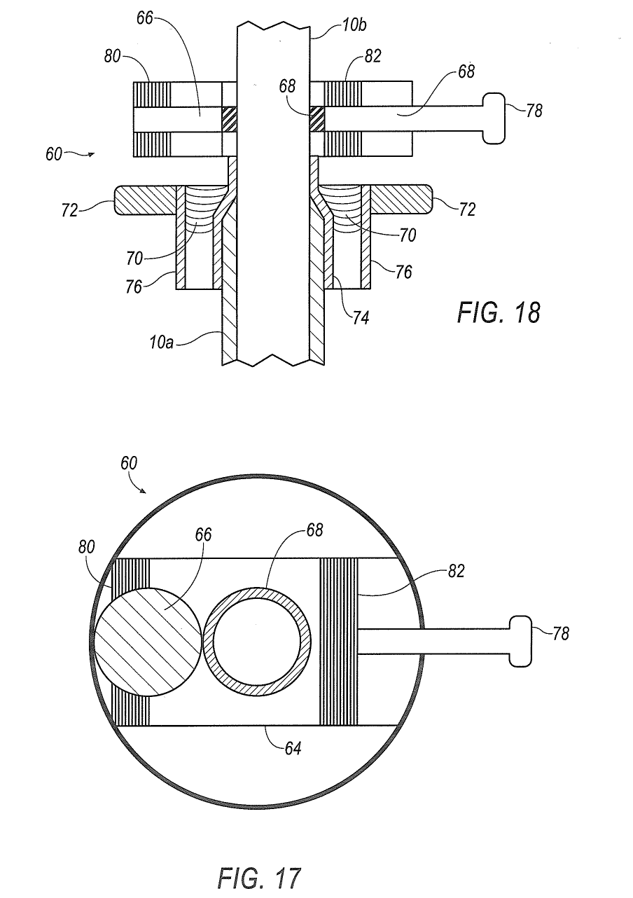

As shown in FIG. 15, when the guard device 60 is attached to sheath 10, the one-way valve 66 may be disposed in a general central region of the guard device 60 and channel 64, such that the one-way valve 66 is generally aligned and disposed with the sheath 10. As can be seen with reference to FIGS. 15 and 16 relative to FIGS. 17 and 18, the guard device 60 may be adjusted (such as via movement of a handle element 78) to move the one-way valve 66 and the hollow lumen one-way valve 68 laterally along the channel 64 of the guard device 60 so as either the one-way value 66 and hollow lumen one-way value 68 can be generally aligned with the sheath 10. The compression springs 80 and 82 bias the one-way valve 66 to the central region of the channel 64. As the handle element 78 is moved relative to the housing 62, the handle element 78 compresses springs 80 and 82 against barrier members 146 at each side of the outer sheath portions 10 and disposed on opposite ends of the channel 64. When the handle element 78 has been moved in this manner, the one-way valve 66 is moved to a valve housing space 67 (FIGS. 15 and 17), which was previously vacant when the one-way valve 66 was centrally positioned with the lumen of the sheath 10. The compression springs 80 and 82 bias the hollow lumen one-way valve 68 towards its home position and assists in frictionally holding the interventional catheter 10b in place and prevent back forces from moving the interventional catheter 10b away from the interventional device 10c and sheath 10.

Thus, the guard device 60 provides two one-way valves to allow the guard and sheath to have multiple roles. For example, the one-way valve 66 may be deployed at the sheath lumen during straight forward interventions that do not require extra backup support. Optionally, the second or hollow lumen one way valve 68 allows for interventions with extra backup support created from the constant positive force on the guide catheter from the controls means 142 and compression springs 80 and 82. As can be seen with reference to FIG. 18, when the second valve 68 is moved to the central portion of the channel 64 and guard device 60, the guide catheter 10b may be inserted through the lumen of the outer sheath portion 10a and the hollow lumen one way valve 68, and may be substantially retained relative to the guard device 60 via the forces applied by the compressed springs 80, 82 and control means 142.

The guard device 60 of the present invention allows a substantial amount of the energy to transmit to the site of the intervention as compared to traditional interventions when the force normally introduce from the operator is always at the proximal end of the sheath and thus away from the site of intervention. This guard device 60 also allows for forward motion and stability of the catheter tip which substantially limits backing away of the catheter tip from the intervention device.

With typical or known sheath or delivery systems, when the end of an intervention device, such as jaws of a device for opening and closing an occlusion or the like, encounter an occlusion or obstruction in the vessel, or when the jaws are opened by the operator, a backward force is created and travels toward the site of least resistance, which is toward the hands of the operator. The sudden large backward force often leads to the interventional catheter being moved backward or away from the intervention site, presenting difficulties in properly intervening the vessel. However, the guard device 60 with its secured to the outer sheath wall 10a and its simultaneous frictional engagement with the interventional catheter 10b and interventional device 10c limits the backward force from separating these components of the delivery system. The guard device 60 thus ultimately limits the effect of the backward force to the operator's hands and provides enhanced control of the intervention device at the intervention site.

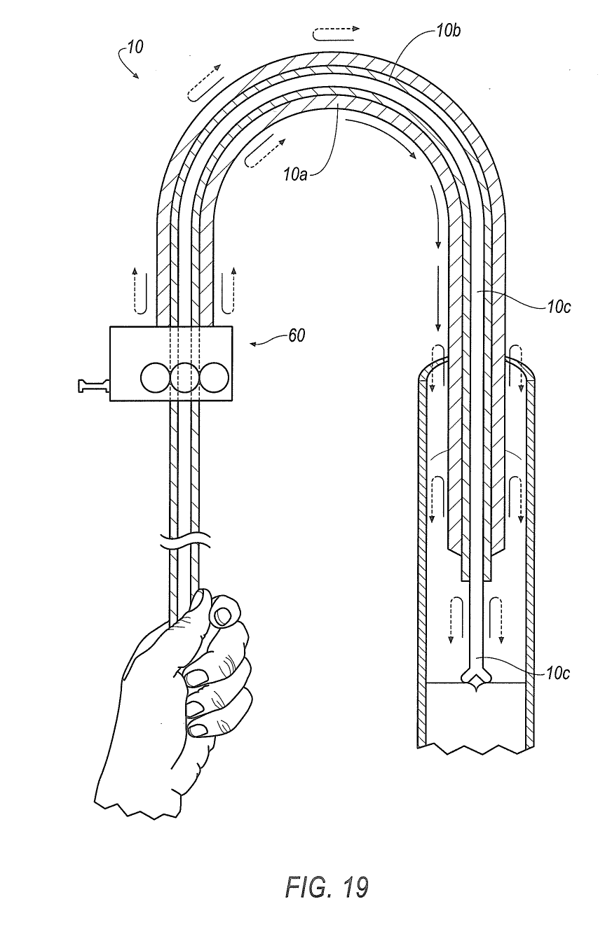

As shown in FIG. 19, the guard device 60 of the present invention allows the point of force to be closer to the intervention site which allows a greater percentage of the force to be maintained on the catheter tip. Typically, when a known intervention device is at an occlusion point, the back force generated travels back on the intervention device and enters the intervention catheter, whereby both backward forces travel backward along the delivery system and toward the operator's hand. As the back force reaches the sheath, which contains both an interventional catheter and device, the second backward force at the catheter may cause the intervention catheter to exit the sheath, which may lead to interventional device failure because the forward force of the device is now canceled by the backward force. However, to improve or maintain the forward force and to prevent the interventional catheter from exiting the sheath, a second source of forward force may be created at the level of the sheath by the guard device of the present invention so as to contain or limit the backward force and redirect the backward force forward along the intervention device.

The guard device 60 of the present invention is thus positioned at the side of the sheath 10 away from the operator and will act as a guard for the energy created from the backward force and maintain the forwardness of the force on the intervention device. This translates into a stable interventional catheter tip and allows the forward force of the intervention device to be transmitted to the intervention site or occlusion, which leads to a higher success rate for such intervention. Additionally, as described above, the sheath 10 may have the varying degrees of flexibility and support due to the varying spacing of the coil springs disposed therealong, the step up end and step down strength of the sheath allows the backward forces to be substantially absorbed as they travel back along the sheath until the proximal end of the sheath (where the springs are condensed and provide a greater strength to the sheath). The combination of the guard device 60 and sheath 10 of the present invention combine to reduce any backward force toward the operator and translate each backward force to forward forces which assists in maintaining a forward pressure at the intervention site.

Therefore, the present invention provides an interventional sheath that provides enhanced flexibility at distal portions of the sheath, while providing enhanced structure rigidity or support at proximal portions of the sheath to assist the physician in moving the sheath through the vessel and providing the desired degree of support at appropriate locations along the vessel. This present invention also provides for enhanced blood flow through the vessel during the intervention procedure, and allows for control of the blood flow via opening and closing of apertures along the sheath during the intervention procedure. Additionally, the present invention provides for a guard device that allows for different one way valves to be disposed at the sheath or catheter and thus limits the backward force generated along the sheath during the intervention procedure to limit the backward force reaching the operator's hands and, thus, to provide enhance control of the intervention device during the intervention procedure.

### Introducer Device for Interventional Sheaths

Optionally, an interventional sheath of the present invention may include an introducer device removably attached to the distal end of the sheath to provide less resistance, improved tracking and greater push ability during interventions crossing Aortoilliac junctions.

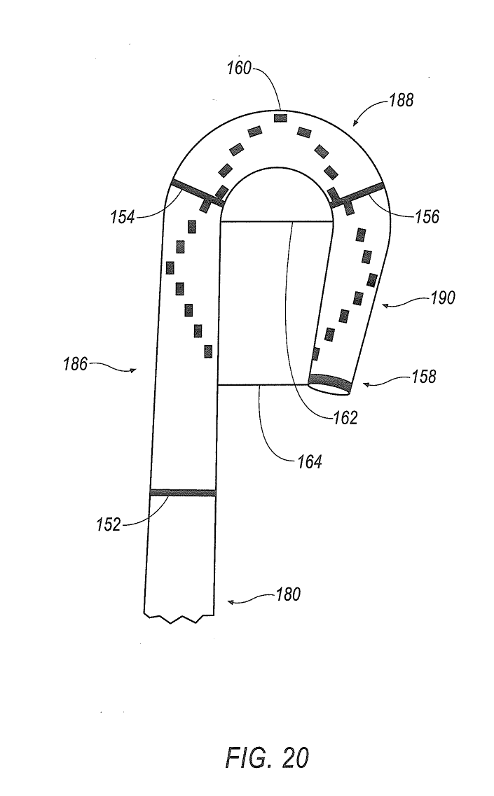

As shown in FIG. 20, the introducer device 150 comprises an extension base 180 that connects to the distal end of the sheath, a first tapered portion 186, a second tapered portion 188, a third tapered portion 190, and a distal tip 158. The introducer device 150 is pre-shaped and biased to be curved at a wide angle 162 to help accommodate interventions through Aortoilliac junctions which is especially beneficial during procedures involving Aortoilliac grafting. The wide angle curve 162 allows the distal tip 158 of the introducer device 150 to be directed medially 164 to deflect flow at the distal tip 158 away from the vessel wall.

The first, second, and third tapered portions 186, 188, 190 of the introducer device 150 vary in diameter, durameter, shape and flexibility. This variability is created by modifying the shape, form, and material used in the distinct regions of the introducer device 150 including the extension base 180 and the first, second, and third tapered portions 186, 188, 190. The first, second, and third tapered portions 186, 188, 190 can be easily and independently modified for differences in desired flexibility and durameter. The shape or curved angle of the introducer device 150 is very flexible with the use of a first angled junction 154 and a second angled junction 156.

In the present example of FIG. 20, the variable durameter allows the introducer device 150 to maneuver its way easily through tortuous vessels without kinking or collapsing. The distal tip 158 will have the smallest diameter and least durameter/hardness of the introducer device 150 portions which allows much needed flexibility at the distal end of the introducer device 150. The extension base 180 will have the greatest diameter and durameter which allows greater rigidity for push-ability and least needed flexibility at the promixate end of the introducer device 150. The first tapered portion 186 will have greater durameter and diameter, and less flexibility than the second tapered portion 188 and the third tapered portion 190. While the second tapered portion 188 will have greater durameter and diameter, and less flexibility than the third tapered portion 190. The introducer device 150 of the current example maintains a range of 25-45 degree's to allow smooth transition over Aortoilliac junctions.

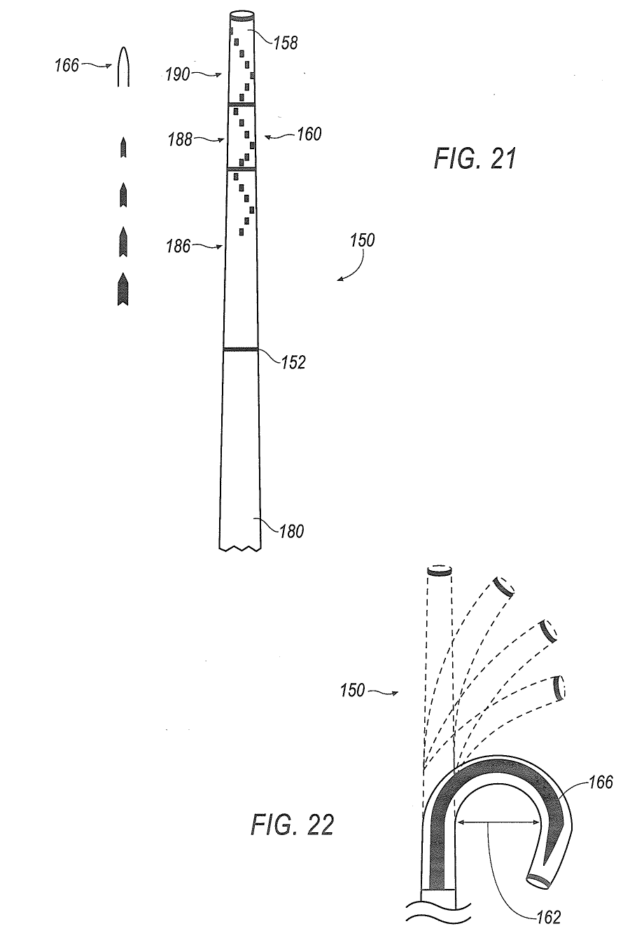

In reference to the example provided in FIGS. 20 and 21, the introducer device 150 may contain perfusion apertures 160 located longitudinally along the introducer device 150 and throughout the first, second, and third tapered portions 186, 188, 190. The introducer device 150 with multiple perfusion apertures allows symmetrical distribution of flow for liquids or contrast media injected into the vessels during the procedure. This symmetrical flow allows better visualization during the intervention for the operator.

In reference to the example provided in FIGS. 20 and 22, the introducer device 150 may also contain a transition zone of curvature in the first tapered portion 186 that the starts at 10-15 cm from the first angle junction 154. The curved wide angle of the introducer device 150 is formed throughout the first, second, and third tapered portions 186, 188, 190. The first angle junction 154 and second angle junction 156 allows easy modification of the shape of the curved introducer device 150. As shown in FIG. 22, the curve 162 can be easily modified to a wide range of angles by simply advancing and retracting a guide wire 166 into the introducer device 150 to force the introducer device 150 into the desired curve or angle. The curved angle 162 of the introducer device 150 can be easily straightened by extending a stiff guide wire 166 all the way through to the distal tip 158 of the introducer device 150. In the example provided by FIG. 22, the introducer device 150 is guided over a 0.35″ guide wire 166. In the example provided in FIG. 22, the curved angle 162 can change within a range of 0-140 degrees. This broad range of angle possibilities allows the operator to engage nearly any vessel including those with the 0-140 degree angles making this introducer device 150 unique and versatile. Once the sheath is advanced to desired position, the introducer device 150 can be removed.

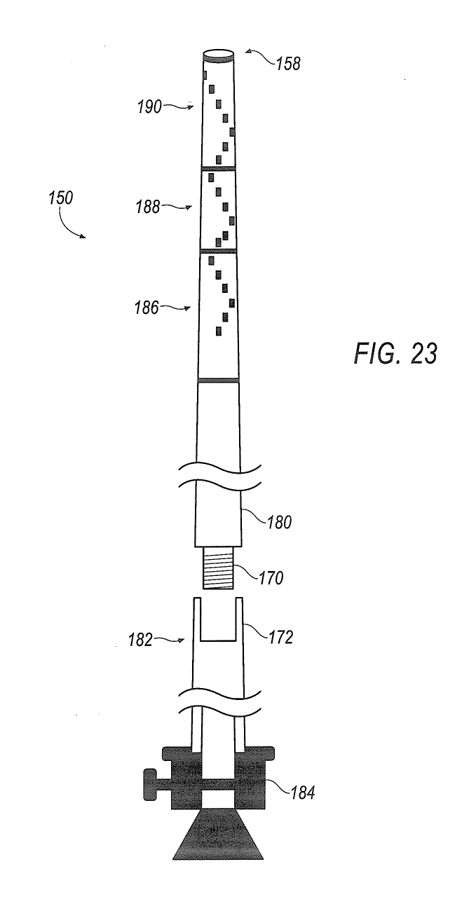

With reference to FIG. 23, an introducer device 150 may be provided and attached to an interventional sheath 182 and guard device 184, such as with the interventional sheath 10 and guard device 60 described above. The introducer device 150 is guided into a patient's vessel over a pre-inserted guide wire. The introducer device 150 may be removably attached at the extension base 180 to a sheath 182. The introducer device 150 is first inserted into a patient's blood vessel while attached to the sheath 182 to aid in the sheath's intervention into remote area of blood vessels. Generally, the introducer device 150 is cylindrically shaped and tapered from the extension base 180 to the distal tip 158. The cylindrical and tapered shape of the introducer device 150 allows a transition of diameters from the thin pre-inserted guide wire to the full diameter of the interventional sheath.

In the example shown in FIG. 23, the introducer device 150 tapers down at the distal tip 158 to a diameter that is 5-10% larger in diameter then a pre-inserted guide wire. The extension base 180 has a similar diameter as the attached sheath 182.

In reference to FIG. 23, the extension base 180 of the introducer device 150 has a male connection member fixedly attached to the proximate end of the extension base 180. The male connection member 170 can be removably fastened to a female connection member 172 which is fixedly attached to the distal end of the sheath 182. As shown in the example of FIG. 23, the fastening means is a simple screw fastening system. A convenient fastening means allows the operator during the intervention procedure to efficiently attach and remove the introducer device 150 from the sheath 182 and guard device 184. However, alternative fastening mechanism known to those skilled in the art can be used to secure the introducer device 150 and the sheath 182 together without departing from the spirit of the invention.

The above descriptions are those of current embodiments of the invention. Various alterations and changes can be made without departing from the broader aspects of the present invention, which is intended to be limited only by the scope of the appended claims, which are to be interpreted in accordance with the principles of patent law including the doctrine of equivalents.

1. An interventional sheath comprising:
a proximal portion having a first degree of flexibility, said proximal portion being at a proximal region of said sheath proximate to the patient entry location;
an intermediate portion having a second degree of flexibility, said intermediate portion being at an intermediation region of said sheath;
a distal portion having a third degree of flexibility, said distal portion being at a distal region of said sheath remote from the patient entry location; and
wherein said second degree of flexibility is greater than said first degree of flexibility, and where said third degree of flexibility is greater than said second degree of flexibility.

2. The interventional sheath of claim 1, wherein said sheath further comprises of support wires for varying the degrees of said flexibility.

3. The interventional sheath of claim 1, wherein said sheath further comprises circular wires at said proximal portion, said intermediate portion and said distal portion, said circular wires having a greater density at said proximal portion than said intermediate portion and having a greater density at said intermediate portion than said distal portion.

4. The interventional sheath of claim 1, wherein said circular wires of said intermediate portion having a density about 50% less than said circular wires of said proximal portion; and
wherein said circular wires of said distal portion having a density about 50% less than said intermediate portion.

5. An interventional sheath comprising:
at least one side aperture at a side wall of said sheath; and
said side aperture being adjustably openable and closable by an operator to control perfusion of blood through said sheath.

6. The interventional sheath of claim 5 further comprising a wire enclosed within said side wall of said sheath, said wire being used to open and close said side apertures.

7. The interventional sheath of claim 6 further comprising at least one side aperture at a side wall of said sheath, said side apertures being openable and closable via movement of said wire within said sheath, said wire having at least one aperture therealong that is alignable with said side aperture at said side wall of said sheath to open said side aperture, said wire being movable to move said at least one aperture away from said side aperture to close said side aperture.

8. The interventional sheath of claim 7, wherein said at least one side aperture at said side wall of said sheath is disposed at said intermediate portion of said sheath.

9. The interventional sheath of claim 5 further comprising an inner circular member enclosed within lumen of said sheath, said inner circular member being used to open and close said side apertures.

10. The interventional sheath of claim 9 further comprising at least one side aperture at a side wall of said sheath, said side apertures being openable and closable via movement of said inner circular member within said sheath lumen, said inner circular member having at least one aperture therealong that is alignable with said side aperture at said side wall of said sheath to open said side aperture, said inner circular member being movable to move said at least one aperture away from said side aperture to close said side aperture.

11. The interventional sheath of claim 9 further comprising a control dial fixedly attached to a proximate portion of said inner circular member and said control dial rotates to control blood perfusion to distal regions of a vessel.

12. The interventional sheath of claim 11 wherein said control dial further comprises:
top portion and bottom portion that engage and hold the inner circular member in a fixed position relative to the sheath;
said bottom portion of said control dial has at least one detent that engage and fit an indent of the top portion; and
wherein said detent is used to intentionally divide the rotation of the control dial into discreet increments that align with the said side apertures of said sheath wall.

13. The interventional sheath of claim 1, wherein said sheath has a length suitable for insertions into the superficial femoral artery and continue to cross and through the horn at the aorto-illiac junction and into the contra-lateral illiac.

14. The interventional sheath of claim 1, wherein said sheath has a length of 80 centimeters.

15. The interventional sheath of claim 1 further comprising:
at least one side aperture at a side wall of said sheath; and
said side aperture being adjustably openable and closable by an operator to control perfusion of blood through said sheath.

16. The interventional sheath of claim 15 further comprising a wire enclosed within said side wall of said sheath, said wire being used to open and close said side apertures.

17. The interventional sheath of claim 15 further comprising an inner circular member enclosed within lumen of said sheath, said inner circular member being used to open and close said side apertures.

18. A guard device for directing an interventional sheath, said guard device comprising:
an outer wall;
a channel extending across said outer wall;
wherein said channel houses a one way valve and a hollow lumen one way value; and
wherein said one-way valve and said hollow lumen one way valve are movable along said channel.

19. The guard device of claim 18, further comprising a threaded region and a collar for rotating said threaded region,
wherein said threaded region of said guard device receives and engages outer wall of said sheath,
wherein threaded region extends along the proximal portion of said sheath, and
rotation of said threaded region causes translational movement of said threaded region along said outer support wall of said sheath.

20. The guard device of claim 18, wherein said guard device further comprising a handle element that communicates and moves said one-way valve and said hollow lumen one way valve along the said channel.

21. The guard device of claim 18, wherein said channel has two compression springs located at each side of said channel; and wherein said compression springs bias the said one-way valve to the center of said channel through communication and adjustable by said handle element.

22. A method for delivering an interventional device to a distal location in a vessel that is remote from a patient entry location, comprising:
providing an interventional sheath comprising a proximal portion having a first degree of flexibility, an intermediate portion having a second degree of flexibility, a distal portion having a third degree of flexibility, and wherein said second degree of flexibility is greater than said first degree of flexibility, and where said third degree of flexibility is greater than said second degree of flexibility; and
wherein said interventional sheath is delivered over a pre-inserted wire assembly.

23. A method for delivering an interventional device to a distal location in a vessel that is remote from a patient entry location, comprising:
providing interventional sheath comprising at least one side aperture at a side wall of said sheath; and
said side aperture being adjustably openable and closable by physician to control perfusion of blood through said sheath.

24. A method for delivering an interventional device to a distal location in a vessel that is remote from a patient entry location, comprising:
providing a guard assembly movably attached to an interventional sheath, said guard assembly providing enhanced control and flexibility of said sheath; and
wherein said guard assembly comprises an outer wall, a channel extending across said outer wall, wherein said channel houses a one way valve, wherein one way value is frictionally engaged with said sheath; and a hollow lumen one way value, wherein said hollow lumen one way valve forcibly retains an interventional catheter.

25. An interventional delivery device comprises:
an interventional sheath for comprising a proximal portion having a first degree of flexibility, an intermediate portion having a second degree of flexibility, a distal portion having a third degree of flexibility, and wherein said second degree of flexibility is greater than said first degree of flexibility, and where said third degree of flexibility is greater than said second degree of flexibility,
at least one side aperture at a side wall of said sheath;
said side aperture being adjustably openable and closable by operator to control perfusion of blood through said sheath; and
a guard device for directing an interventional sheath, said guard device comprising, an outer wall, a channel extending across said outer wall, wherein said channel houses a one way valve and a hollow lumen one way value, and wherein said one-way valve and said hollow lumen one way valve are movable along said channel.

26. An introducer device for an interventional sheath comprising:
a cylinder housing; wherein said housing contains a plurality of engaged portions;
a base; wherein said base is capable of fastening to the distal end of an interventional sheath; and
a distal tip.

27. An introducer device of claim 26, wherein said cylinder housing is tapered from said base to said distal tip.

28. An introducer device of claim 27, wherein said plurality of engaged portions further comprises:
a proximal portion having a first degree of flexibility, said proximal portion being at a proximal region of said introducer device proximate to the patient entry location;
an intermediate portion having a second degree of flexibility, said intermediate portion being at an intermediation region of said introducer device;
a distal portion having a third degree of flexibility, said distal portion being at a distal region of said introducer device remote from the patient entry location; and
wherein said second degree of flexibility is greater than said first degree of flexibility, and where said third degree of flexibility is greater than said second degree of flexibility.

29. An introducer device of claim 27, wherein said plurality of engaged portions are pre-shaped to form a curve that has a wide based angle and wherein said distal tip is medially directed towards said cylinder housing.

30. An introducer device of claim 27, wherein said cylinder housing further comprises at least one side aperture in a side wall of said cylinder housing.

31. An introducer device of claim 27, wherein said cylinder housing further comprises a hollow lumen aligned longitudinally therealong, wherein said hollow lumen is adapted to receive a pre-inserted guide wire.

32. A method for delivering an interventional device to a distal location in a vessel that is remote from a patient entry location, comprising:
providing a introducer device movably attached to an interventional sheath; and said introducer device providing enhanced control and flexibility of said sheath.

33. A method for delivering an interventional device to a distal location in a vessel that is remote from a patient entry location, comprising:
providing an introducer device;
wherein said introducer device comprises,
a cylindrically tapered housing, wherein said housing contains a plurality of engaged portions,
a base, wherein said base is capable of fastening to the distal end of an interventional sheath;
a distal tip; and
wherein said introducer device is delivered over a pre-inserted wire assembly.

34. A method for delivering an interventional device according to claim 33 wherein said cylindrically tapered housing further comprises:
a proximal portion having a first degree of flexibility;
an intermediate portion having a second degree of flexibility;
a distal portion having a third degree of flexibility; and
wherein said second degree of flexibility is greater than said first degree of flexibility, and where said third degree of flexibility is greater than said second degree of flexibility.

35. A method for delivering an interventional device according to claim 33 wherein said cylindrically tapered housing further comprises at least one side aperture at a side wall of said cylindrically tapered housing.

36. An interventional delivery device comprises:
an interventional sheath for comprising a proximal portion having a first degree of flexibility, an intermediate portion having a second degree of flexibility, a distal portion having a third degree of flexibility, and wherein said second degree of flexibility is greater than said first degree of flexibility, and where said third degree of flexibility is greater than said second degree of flexibility;
at least one side aperture at a side wall of said sheath;
said side aperture being adjustably openable and closable by operator to control perfusion of blood through said sheath;
a guard device for securing an interventional sheath, said guard device comprising, an outer wall, a channel extending across said outer wall, wherein said channel houses a one way valve and a hollow lumen one way value, and wherein said one-way valve and said hollow lumen one way valve are movable along said channel; and
an introducer device for directing an intervention sheath, said introducer device comprising, a cylindrically tapered housing, wherein said housing contains a plurality of engaged portions, a base, wherein said base is removably attached to the distal end of an interventional sheath.
